(19) United States
(12) Patent Application Publication
(10) Pub. No.: US 2003/0158169 A1
(43) Pub. Date: Aug. 21, 2003

(54) Method of treating hemorrhagic shock or systemic inflammatory response syndrome

(75) Inventors: Heather A. Harris, Phoenixville, PA (US); Ruth A. Henderson, Swarthmore, PA (US)

Correspondence Address: Arnold S. Milowsky, Five Giralda Farms, Madison, NJ, 07940, US

(73) Assignee: Wyeth, Madison, NJ

(21) Appl. No.: 10/348,511

(22) Filed: Jan. 21, 2003

Related U.S. Application Data
(60) Provisional application No. 60/352,115, filed on Jan. 24, 2002.

(51) Int. Cl. A61K 31/56

(52) U.S. Cl. 514/182

(57) ABSTRACT

This invention provides the a method of treating or inhibiting hemorrhagic shock or systemic inflammatory response syndrome using an ERβ selective ligand.

This application claims priority from copending provisional application Serial No. 60/352,115, filed Jan. 24, 2002, the entire disclosure of which is hereby incorporated by reference.

### BACKGROUND OF THE INVENTION

This invention relates to a method of using ERβ selective ligands in the treatment of hemorrhagic shock or systemic inflammatory response syndrome (SIRS).

SIRS is a term defined in 1991 to aid in the classification of sepsis and its sequelae (for review, see [Fry (2000) American Surgeon 66, 126-32.]). As the name implies, it is a global immune response. Any number of bodily insults can elicit this inflammatory response: Trauma, invasive infection, failure of host defense mechanisms, burns or hemorrhagic shock. The prevalence of SIRS is extremely high, for example it has been estimated that more than 80% of surgical patients in intensive care units have this syndrome [Brun-Buisson (2000) Intensive Care Medicine 26, S64-74.].

Hemorrhagic shock is caused by acute blood loss of at least 20% of normal blood volume. This reduced volume of circulating blood causes tissue hypoperfusion and leads to local ischemia and damage. Ultimately hypovolemic shock can lead to multiple system organ failure [Keith (1986) Circulatory Shock 19, 283-92.]. When lost fluids are replaced (reperfusion) in an attempt to stabilize blood pressure additional damage can occur.

The gastrointestinal tract is especially sensitive to hemorrhagic shock and reperfusion. Necrosis occurs and bacterial endotoxin release are commonly observed [Tamion, et al. (1997) American Journal of Physiology 273, G314-21.]. In response to the infiltration of bacteria into the blood stream, a maelstrom of proinflammatory cytokines can be released, contributing to multiple organ failure and development of SIRS [Tamion, et al. (1997) American Journal of Physiology 273, G314-21, Rowlands, et al. (1999) British Medical Bulletin 55, 196-211, Hassoun, et al. (2001) Shock 15, 1-10, Pape, et al. (1994) British Journal of Surgery 81, 850-2.]. Thus, hemorrhagic shock patients are at increased risk of developing SIRS.

Gender differences in response to various preclinical models of trauma-hemorrhage and sepsis have been described, with males having increased mortality and morbidity compared to females [Diodato, et al. (2001) Cytokine 14, 162-169, Angele, et al. (2000) Shock 14, 81-90.]. One of the contributing factors to the apparent protection of females from these types of insults seems to be estrogen. For example, treatment of males with estrogen improves cardiac output and liver function [Mizushima, et al. (2000) Annals of Surgery 232, 673-679.] and ovariectomy of reduces the immunocompetence of females [Knoferl, et al. (2001) American Journal of Physiology Cell Physiology 281, C1131-C1138.] following insult. Additionally, administration of an estrogen receptor antagonist blocks the beneficial effects of estrogen [Jarrar, et al. (2000) Surgery 128, 246-252.].

Estrogens have previously been shown to modulate the immune system both though direct effect on immune system cells, e.g. cell survival, (for review see [Cutolo, et al. (1995) Clinical & Experimental Rheumatology 13, 217-26, Jansson and Holmdahl (1998) Inflammation Research 47, 290-301.]) and though influencing cytokine production via NF-KB inhibition [Ray, et al. (1994) Journal of Biological Chemistry 269, 12940-6, Stein and Yang (1995) Molecular & Cellular Biology 15, 4971-9.]. Additionally, in an in vivo model of trauma and hemorrhagic shock, Th1 cytokine release by male splenocytes was maintained by estrogen administration [Angele, et al. (2001) Cytokine 16, 22-30.] while circulating levels of IL-6 were reduced [Knoferl, et al. (2000) Archives of Surgery 135, 425-433.].

Estrogens exert their actions in cells by binding to receptors, of which two are known. The second form of the estrogen receptor (ER) was recently discovered [Tremblay, et al. (1997) Molecular Endocrinology 11, 353-365, Bhat, et al. (1998) Journal of Steroid Biochemistry & Molecular Biology 67, 233-240, Kuiper, et al. (1996) Proceedings of the National Academy of Sciences of the United States of America 93, 5925-5930, Mosselman, et al. (1996) FEBS Letters 392, 49-53.], and this protein has been designated ERβ to distinguish it from the previously known form, now called ERα. Although the tissue distribution [Brandenberger, et al. (1997) Journal of Clinical Endocrinology & Metabolism 82, 3509-12, Kuiper, et al. (1997) Endocrinology 138, 863-870, Saji, et al. (2000) Proceedings of the National Academy of Sciences of the United States of America 97, 337-342, Saunders, et al. (1997) Journal of Endocrinology 154, R13-R16, Shughrue, et al. (1997) Journal of Comparative Neurology 388, 507-525, Taylor and Al-Azzawi (2000) Journal of Molecular Endocrinology 24, 145-155, Vidal, et al. (1999) Journal of Bone & Mineral Research 14, 923-929, Wang, et al. (2000) Biology of Reproduction 63, 1331-1340.], ligand specificity [Kuiper, et al. (1997) Endocrinology 138, 863-870, Kuiper, et al. (1998) Endocrinology 139, 42524263.] and three-dimensional structure (through X-ray crystallography) [Pike, et al. (1999) EMBO Journal 18, 4608-4618.] of ERβ have been studied, defining its function has remained elusive.

Of interest to this invention, is the observation that ERβ can interfere with NF-κB transcriptional activation, as evaluated using the non-receptor selective estrogen, 17β-estradiol [Bhat, et al. (1998) Journal of Steroid Biochemistry & Molecular Biology 67, 233-240, Harnish, et al. (2000) Endocrinology 141, 3403-11.]. In addition, ERβ is expressed in cells of the immune system [Mosselman, et al. (1996) FEBS Letters 392, 49-53, Brandenberger, et al. (1997) Journal of Clinical Endocrinology & Metabolism 82, 3509-12, Rider, et al. (2000) Clinical Immunology 95, 124-134.].

### DESCRIPTION OF THE INVENTION

This invention provides the use of a ERβ selective ligand in the treatment or inhibition of hemorrhagic shock or systemic inflammatory response syndrome.

As used in accordance with this invention, the term “treatment” means treating a mammal having or being susceptible to hemorrhagic shock or systemic inflammatory response syndrome by providing said mammal an effective amount of a ERβ selective ligand with the purpose of inhibiting onset or progression of the hemorrhagic shock or systemic inflammatory response syndrome. These conditions can be caused by, for example, severe injury (gunshot, knife wound, surgery, automobile accident, burns) or infections (intestinal perforations, breakdown of intestinal permeability barrier, aseptic surgical procedures, puncture wounds).

As used in accordance with this invention, the term “providing” means either directly administering the ERβ selective ligand, or administering a prodrug, derivative, or analog of the ERβ selective ligand that will form an effective amount of the ERβ selective ligand within the body.

As used in accordance with this invention, the term “ERβ selective ligand” means that the binding affinity (as measured by IC₅₀, where the IC₅₀ of 17β-estradiol is not more than 3 fold different between ERα and ERβ) of the ligand to ERβ is at least about 10 times greater than its binding affinity to ERα in a standard pharmacological test procedure that measures the binding affinities to ERα and ERβ. It is preferred that the ERβ selective ligand will have a binding affinity to ERβ that is at least about 20 times greater than its binding affinity to ERα. It is more preferred that the ERβ selective ligand will have a binding affinity to ERβ that is at least about 50 times greater than its binding affinity to ERα. It is further preferred that the ERβ selective ligand is non-uterotrophic and non-mammotrophic.

As used in accordance with this invention, the term “non-uterotrophic” means producing an increase in wet uterine weight in a standard pharmacological test procedure of less than about 50% of the uterine weight increase observed for a maximally efficacious dose of 17β-estradiol or 17α-ethinyl-17β-estradiol in the same procedure. It is preferred that the increase in wet uterine weight will be less than about 25% of that observed for estradiol, and more preferred that the increase in wet uterine weight will be less than about 10% of that observed for estradiol. It is most preferred that the non-uterotrophic ERβ selective ligand will not increase wet uterine weight significantly (p<0.05) compared with a control that is devoid of uterotrophic activity (e.g. vehicle).

As used in accordance with this invention, the term “non-mammotrophic” means producing an increase in casein kinase II mRNA in a standard pharmacological test procedure of less than about 50% of the casein kinase II mRNA increase observed for a maximally efficacious dose of 17β-estradiol or 17α-ethinyl-17β-estradiol in the same procedure. It is preferred that the increase casein kinase II mRNA will be less than about 25% of that observed for estradiol, and more preferred that the increase in casein kinase II mRNA will be less than about 10% of that observed for estradiol. It is most preferred that the non-mammotrophic ERβ selective ligand will not increase casein kinase II mRNA significantly (p<0.05) compared with a control that is devoid of mammotrophic activity (e.g. vehicle).

Evaluation of Binding Affinities to ERα and ERβ

Compounds can be evaluated for their ability to compete with 17β-estradiol using both ERα and ERβ. This test procedure provides the methodology for one to determine the relative binding affinities for the ERα or ERβ receptors. The procedure used is briefly described below.

Preparation of receptor extracts for characterization of binding selectivity. The ligand binding domains, conveniently defined here as all sequence downstream of the DNA binding domain, are obtained by PCR using full length cDNA as templates and primers that contained appropriate restriction sites for subcloning while maintaining the appropriate reading frame for expression. These templates contain amino acids M₂₅₀-V₅₉₅ of human ERα [Green, et al. (1986) Nature 320, 134-9.] and M₂₁₄-Q₅₃₀ of human ERβ [Ogawa, et al. (1998) Biochemical & Biophysical Research Communications 243, 122-6.]. Human ERβ is cloned into pET15b (Novagen, Madison Wis.) as a Nco1-BamH1 fragment bearing a C-terminal Flag tag. Human ERα is cloned as for human ERβ except that an N-terminal His tag is added. The sequences of all constructs used are verified by complete sequencing of both strands.

BL21(DE3) cells are used to express the human proteins. Typically a 10 mL overnight culture is used to inoculate a 1 L culture of LB medium containing 100 μg/mL of ampicillin. After incubation overnight at 37° C., IPTG is added to a final concentration of 1 mM and incubation proceeded at 25° C. for 2 hours. Cells are harvested by centrifugation (1500×g), and the pellets washed with and resuspended in 100 mL of 50 mM Tris-Cl (pH 7.4), 150 mM NaCl. Cells are lysed by passing twice through a French press at 12000 psi. The lysate is clarified by centrifugation at 12,000×g for 30 minutes at 4° C. and stored at −70° C.

Evaluation of extracts for specific [³H]-estradiol binding. Dulbecco's phosphate buffered saline (GibcoBRL, Grand Island N.Y., 1× final concentration) supplemented with 1 mM EDTA is used as the assay buffer. To optimize the amount of receptor to use in the assay, [³H]-17β-estradiol (New England Nuclear; final concentration=2 nM)±0.6 μM diethlystilbestrol and 100 μL of various dilutions of the E. coli lysate are added to each well of a high binding masked microtiter plate (EG&G Wallac/PE Life Sciences, Boston, Mass.). The final assay volume is 120 μL and the concentration of DMSO is ≦1%. After incubation at room temperature for 5-18 hours, unbound material is aspirated and the plate washed three times with approximately 300 μL of assay buffer. After washing, 135 μL of scintillation cocktail (Optiphase Supermix, EG&G Wallac/PE Life Sciences, Boston, Mass.) is added to the wells, and the plate is sealed and agitated for at least 5 minutes to mix scintillant with residual wash buffer. Bound radioactivity is evaluated by liquid scintillation counting (EG&G Wallac Microbeta Plus).

After determining the dilution of each receptor preparation that provided maximum specific binding, the assay is further optimized by estimating the IC₅₀ of unlabelled 17β-estradiol using various dilutions of the receptor preparation. A final working dilution for each receptor preparation is chosen for which the IC₅₀ of unlabelled 17β-estradiol is 2-4 nM.

Ligand binding competition test procedure. Test compounds are initially solubilized in DMSO and the-final concentration of DMSO in the) binding assay is ≦1%. Eight dilutions of each test compound are used as an unlabelled competitor for [³H]-17β-estradiol. Typically, a set of compound dilutions would be tested simultaneously on human ERα and ERβ. The results are plotted as measured DPM vs. concentration of test compound. For dose-response curve fitting, a four parameter logistic model on the transformed, weighted data is fit and the IC₅₀ is defined as the concentration of compound decreasing maximum [³H]-estradiol binding by 50%.

Evaluation of Uterotrophic Activity

Uterotrophic activity of a test compound can be measured according to the following standard pharmacological test procedure. Sexually immature (18 days of age) 129 SvE mice are obtained from Taconic Farms (Germantown, N.Y.) and provided unrestricted access to a casein-based diet (Purina Mills 5K96C) and water. On day 22, 23, 24 and 25 the mice are dosed subcutaneously with compound or vehicle (corn oil). There are six mice/group and they are euthanized approximately 6 hours after the last injection by CO₂ asphyxiation and pneumothorax. Uteri are removed and weighed after trimming associated fat and expressing any internal fluid.

The sexually immature mouse uterus expresses primarily ERα [[Couse, et al. (1997) Endocrinology 138, 4613-4621.]] and is highly responsive to estrogens. Upon stimulation with estrogen the mouse uterus typically increases in size three to four fold and coadministration with a receptor antagonist can block this effect.

Evaluation in the Mammary End Bud Test Procedure

Estrogens are required for full ductal elongation and branching of the mammary ducts, and the subsequent development of lobulo-alveolar end buds under the influence of progesterone. In this test procedure, the mammotrophic activity of ER-β selective compounds can be evaluated according to the following standard pharmacological test procedure. Twenty-eight day old Sprague-Dawley rats (Taconic Farms, Germantown, N.Y.) are ovariectomized and rested for nine days. Animals are housed under a 12-hour light/dark cycle and fed a casein-based Purina Laboratory Rodent Diet 5K96 (Purina, Richmond, Ind.) and water ad libidum. Rats are then dosed subcutaneously for six days with vehicle (50% DMSO (J T Baker, Phillipsburg, N.J.)/50% 1×Dulbecco's Phosphate buffered saline (GibcoBRL, Grand Island, N.Y.), 17β-estradiol (0.1 mg/kg) or an ER-β selective ligand (various doses). For the final three days, rats are also dosed subcutaneously with progesterone (30 mg/kg). On the seventh day, rats are euthanised and a mammary fat pad excised. This fat pad is analyzed for casein kinase II mRNA as a marker of end bud proliferation. Casein kinase II mRNA is anlayzed by real-time RT-PCR. Briefly, RNA is isolated following Trizol (GibcoBRL, Grand Island, N.Y.) according to the manufacture's directions, Samples are treated with DNAse I using DNA-free kit (Ambion), and casein kinase II mRNA levels are measured by real-time RT-PCR using the Taqman Gold procedure (PE Applied Biosystems). A total of 50 ng of RNA is analyzed in triplicate using casein kinase II specific primer pair (5′ primer, CACACGGATGGCGCATACT; 3′ primer, CTCGGGATGCACCATGAAG) and customized probe (TAMRA-CGGCACTGGTTTCCCTCACATGCT-FAM). Casein kinase II mRNA levels are normalized to 18s ribosomal RNA contained within each sample reaction using primers and probe supplied by PE Applied Biosystems.

Evaluation in the Hemorrhagic Shock (With or Without Sepsis) Test Procedure

This standard pharmacological test procedure is described in the literature [Diodato, et al. (2001) Cytokine 14, 162-169, Knoferl, et al. (2001) American Journal of Physiology Cell Physiology 281, C1131-C1138.]. Briefly, a rodent (mouse or rat) is subjected to soft tissue trauma and controlled surgical hemorrhage followed by fluid resuscitation. Sepsis can also be induced during surgery by cecal ligation and puncture.

Evaluation in the Intestinal Ishemia/Reperfusion Injury Test Procedure

This standard pharmacological test procedure is described in the literature [Du, et al. (1997) American Journal of Physiology 272, G545-52.]. Briefly the superior mesenteric artery is occluded for 90 minutes followed by reperfusion.

Evaluation in the Pseudomonas-Induced Sepsis Test Procedure

This standard pharmacological test procedure is described in the literature [Opal, et al. (1998) Journal of Infectious Diseases 178, 1205-8.]. In this model, neutropenic mice are orally administered Pseudomonas bacteria which readily colonize the gut leading to systemic infection and certain death. Test compounds can increase survival when administered at the onset of fever.

Evaluation in the Mouse Burn Test Procedure

This standard pharmacological test procedure is described in the literature [Schindel, et al. (1997) Journal of Pediatric Surgery 32, 312-5.]. Anesthetized mice are subjected to a full skin thickness burn over 32% of their body. Septic inflammatory response syndrome develops as bacteria enter the body not only through the injured skin tissue but also through the intestine, whose permeability is increased by trauma.

Evaluation in the Listeria Monocytogenes Challenge Test Procedure

This standard pharmacological test procedure is described in the literature [Opal, et al. (2000) J Infectious Diseases 181, 754-756.]. Briefly, mice are challenged with an LD50 dose of Listeria in the presence or absence of test compound. The effect of test compound on Listeria-induced mortality is measured. Other endpoints include cytokine levels tissue distribution of the Listeria pathogen.

When administered for the treatment or inhibition of a particular disease state or disorder, it is understood that the effective dosage may vary depending upon the particular compound utilized, the mode of administration, the condition, and severity thereof, of the condition being treated, as well as the various physical factors related to the individual being treated. It is projected that effective administration of the compounds of this invention may be given at a daily oral dose of from about 5 μg/kg to about 100 mg/kg. The projected daily dosages are expected to vary with route of administration, and the nature of the compound administered.

Such doses may be administered in any manner useful in directing the active compounds herein to the recipient's bloodstream, including orally, via implants, parenterally (including intravenous, intraperitoneal and subcutaneous injections), intraarticularly, rectally, intranasally, intraocularly, vaginally, or transdermally.

Oral formulations containing the active compounds of this invention may comprise any conventionally used oral forms, including tablets, capsules, buccal forms, troches, lozenges and oral liquids, suspensions or solutions. Capsules may contain mixtures of the active compound(s) with inert fillers and/or diluents such as the pharmaceutically acceptable starches (e.g. corn, potato or tapioca starch), sugars, artificial sweetening agents, powdered celluloses, such as crystalline and microcrystalline celluloses, flours, gelatins, gums, etc. Useful tablet formulations may be made by conventional compression, wet granulation or dry granulation methods and utilize pharmaceutically acceptable diluents, binding agents, lubricants, disintegrants, surface modifying agents (including surfactants), suspending or stabilizing agents, including, but not limited to, magnesium stearate, stearic acid, talc, sodium lauryl sulfate, microcrystalline cellulose, carboxymethylcellulose calcium, polyvinylpyrrolidone, gelatin, alginic acid, acacia gum, xanthan gum, sodium citrate, complex silicates, calcium carbonate, glycine, dextrin, sucrose, sorbitol, dicalcium phosphate, calcium sulfate, lactose, kaolin, mannitol, sodium chloride, talc, dry starches and powdered sugar. Preferred surface modifying agents include nonionic and anionic surface modifying agents. Representative examples of surface modifying agents include, but are not limited to, poloxamer 188, benzalkonium chloride, calcium stearate, cetostearl alcohol, cetomacrogol emulsifying wax, sorbitan esters, colloidol silicon dioxide, phosphates, sodium dodecylsulfate, magnesium aluminum silicate, and triethanolamine. Oral formulations herein may utilize standard delay or time release formulations to alter the absorption of the active compound(s). The oral formulation may also consist of administering the active ingredient in water or a fruit juice, containing appropriate solubilizers or emulsifiers as needed.

In some cases it may be desirable to administer the compounds directly to the airways in the form of an aerosol.

The compounds of this invention may also be administered parenterally (such as directly into the joint space) or intraperitoneally. Solutions or suspensions of these active compounds as a free base or pharmacologically acceptable salt can be prepared in water suitably mixed with a surfactant such as hydroxy-propylcellulose. Dispersions can also be prepared in glycerol, liquid polyethylene glycols and mixtures thereof in oils. Under ordinary conditions of storage and use, these preparation contain a preservative to prevent the growth of microorganisms.

The pharmaceutical forms suitable for injectable use include sterile aqueous solutions or dispersions and sterile powders for the extemporaneous preparation of sterile injectable solutions or dispersions. In all cases, the form must be sterile and must be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (e.g., glycerol, propylene glycol and liquid polyethylene glycol), suitable mixtures thereof, and vegetable oils.

For the purposes of this disclosure, transdermal administrations are understood to include all administrations across the surface of the body and the inner linings of bodily passages including epithelial and mucosal tissues. Such administrations may be carried out using the present compounds, or pharmaceutically acceptable salts thereof, in lotions, creams, foams, patches, suspensions, solutions, and suppositories (rectal and vaginal).

Transdermal administration may be accomplished through the use of a transdermal patch containing the active compound and a carrier that is inert to the active compound, is non toxic to the skin, and allows delivery of the agent for systemic absorption into the blood stream via the skin. The carrier may take any number of forms such as creams and ointments, pastes, gels, and occlusive devices. The creams and ointments may be viscous liquid or semisolid emulsions of either the oil-in-water or water-in-oil type. Pastes comprised of absorptive powders dispersed in petroleum or hydrophilic petroleum containing the active ingredient may also be suitable. A variety of occlusive devices may be used to release the active ingredient into the blood stream such as a semi-permeable membrane covering a reservoir containing the active ingredient with or without a carrier, or a matrix containing the active ingredient. Other occlusive devices are known in the literature.

Suppository formulations may be made from traditional materials, including cocoa butter, with or without the addition of waxes to alter the suppository's melting point, and glycerin. Water soluble suppository bases, such as polyethylene glycols of various molecular weights, may also be used.

What is claimed is:

1. A method of treating or inhibiting hemorrhagic shock in a mammal in need thereof, which comprises providing to said mammal an effective amount of a non-uterotropic, non-mammotrophic ERβ selective ligand.

2. The method according to claim 1, wherein the binding affinity of the ERβ selective ligand to ERβ is at least about 20 times greater than its binding affinity to ERα.

3. The method according to claim 2, wherein the binding affinity of the ERβ selective ligand to ERβ is at least about 50 times greater than its binding affinity to ERα.

4. The method according to claim 3, wherein the ERβ selective ligand causes an increase in wet uterine weight is less than about 25% of that observed for a maximally efficacious dose of 17β-estradiol in a standard pharmacological test procedure measuring uterotrophic activity.

5. The method according to claim 4, wherein the ERβ selective ligand causes an increase in casein kinase II mRNA which is less than about 25% of that observed for a maximally efficacious dose of 17β-estradiol in a standard pharmacological test procedure measuring mammotrophic activity.

6. The method according to claim 5, wherein the ERβ selective ligand causes an increase in wet uterine weight which is less than about 10% of that observed for a maximally efficacious dose of 17β-estradiol in a standard pharmacological test procedure measuring uterotrophic activity.

7. The method according to claim 6, wherein the ERβ selective ligand causes an increase in casein kinase II mRNA which is less than about 10% of that observed for a maximally efficacious dose of 17β-estradiol in a standard pharmacological test procedure measuring mammotrophic activity.

8. The method according to claim 7, wherein the ERβ selective ligand does not significantly (p<0.05) increase wet uterine weight compared with a control that is devoid of uterotrophic activity, and does not significantly (p<0.05) increase casein kinase II mRNA compared with a control that is devoid of mammotrophic activity.

9. A method of treating or inhibiting systemic inflammatory response syndrome in a mammal in need thereof, which comprises providing to said mammal an effective amount of a non-uterotropic, non-mammotrophic ERβ selective ligand.

10. The method according to claim 9, wherein the binding affinity of the ERβ selective ligand to ERβ is at least about 20 times greater than its binding affinity to ERα.

11. The method according to claim 10, wherein the binding affinity of the ERβ selective ligand to ERβ is at least about 50 times greater than its binding affinity to ERα.

12. The method according to claim 11, wherein the ERβ selective ligand causes an increase in wet uterine weight is less than about 25% of that observed for a maximally efficacious dose of 17β-estradiol in a standard pharmacological test procedure measuring uterotrophic activity.

13. The method according to claim 12, wherein the ERβ selective ligand causes an increase in casein kinase II mRNA which is less than about 25% of that observed for a maximally efficacious dose of 17β-estradiol in a standard pharmacological test procedure measuring mammotrophic activity.

14. The method according to claim 13, wherein the ERβ selective ligand causes an increase in wet uterine weight which is less than about 10% of that observed for a maximally efficacious dose of 17β-estradiol in a standard pharmacological test procedure measuring uterotrophic activity.

15. The method according to claim 14, wherein the ERβ selective ligand causes an increase in casein kinase II mRNA which is less than about 10% of that observed for a maximally efficacious dose of 17β-estradiol in a standard pharmacological test procedure measuring mammotrophic activity.

16. The method according to claim 15, wherein the ERβ selective ligand does not significantly (p<0.05) increase wet uterine weight compared with a control that is devoid of uterotrophic activity, and does not significantly (p<0.05) increase casein kinase II mRNA compared with a control that is devoid of mammotrophic activity.

17. A method of treating or inhibiting organ damage or failure resulting from hemorrhagic shock or systemic inflammatory response syndrome in a mammal in need thereof, which comprises providing to said mammal an effective amount of a non-uterotropic, non-mammotrophic ERβ selective ligand.

18. A method of treating or inhibiting tissue damage following hypoperfusion in a mammal with low blood volume, which comprises providing to said mammal an effective amount of a non-uterotropic, non-mammotrophic ERβ selective ligand.

19. A method of inhibiting an increase in intestinal permiability following traumatic injury in a mammal in need thereof, which comprises providing to said mammal an effective amount of a non-uterotropic, non-mammotrophic ERβ selective ligand.
